(19) United States
(12) Patent Application Publication
(10) Pub. No.: US 2009/0047350 A1
(43) Pub. Date: Feb. 19, 2009

(54) Perforated water soluble polymer based edible films

(76) Inventor: Ramesh Bangalore, Pleasanton, CA (US)

Correspondence Address: RAMESH BANGALORE, 5274 RIVERDALE CT, Pleasanton, CA, 94588, US

(21) Appl. No.: 12/228,703

(22) Filed: Aug. 14, 2008

Related U.S. Application Data
(60) Provisional application No. 60/965,043, filed on Aug. 17, 2007.
(60) Provisional application No. 60/965,023, filed on Aug. 17, 2007.

(51) Int. Cl. A61K 9/14; A61K 31/496; A61P 15/00

(52) U.S. Cl. 424/486; 514/252.16

(57) ABSTRACT

A novel edible polymer based film dosage form manufactured using natural, synthetic, semisynthetic, pharmaceutically acceptable polymers addressing the issues of swallowing difficulties (Dysphagia and Dynaphagia), of tablet or capsule dosage forms and handling and storage difficulties associated with liquid dosage forms, that also includes materials such as emulsifying agents, suspending agents, buffering agents, effervescence agents, colorants, flavorants, sweetners and specified amounts of bioactive agents preferably having perforations in the body of the dosage form enabling it to disperse/dissolve rapidly upon application by the subject. A flexible film dosage form to accommodate higher drug loads without compromising the fast dispersing/dissolving characteristics is presented. The perforated film system is enabled to be used in various applications such as oral, mucosal and external environments. The perforated film dosage form therefore, additionally allows the usage of polymers that would otherwise have not been used due to insolubility or limited solubility in water.

### CLAIM OF PROVISIONAL APPLICATION RIGHTS

This application claims the benefit of U.S. Provisional Patent Application Nos. 60/965,043 and 60/965,023 filed on Aug. 17, 2007.

### TECHNICAL FIELD OF INVENTION

The present invention related to drug delivery systems and more specifically it is related to administration of bioactive agents via perforated orally fast dispersing/dissolving polymer based films in the biological system.

### BACKGROUND OF INVENTION

Some patients experience difficulty in swallowing tablets and capsules, leading to problems in administration of dosage forms. This physiological condition is known as “Dysphagia”, difficulty in swallowing or “Dynophagia”, painful swallowing. Such conditions are often experienced by older patients, patients with head/neck injuries or AIDS patients. It is also a common experience with pediatric patients who tend to be non compliant with the administration of solid oral dosage forms. To address the issue of delivery of drugs to such patients liquid dosage forms have been developed. However, handling and delivering the liquid systems is always a challenge. Therefore, dosage forms with the convenience of liquids and the dose precision of solid oral dosage form is desirable. Additionally, dosage forms that do not require consumption of liquids for administration exclusively are desirable for drug administration to dialysis patients who have the restriction on fluids consumption. This feature in dosage forms makes dose administration convenient even for patients on the go improving overall patient compliance with treatment regimens.

In addition, the conventional oral dosage forms, such as tablets, pills, caplets, and capsules, are designed for short residence time in the mouth. Absorption of the active agent from these dosage forms occurs in the gastrointestinal (GI) tract only after the dosage form undergoes disintegration followed by dissolution of the agent in the gastric fluids. However, for some active agents, it is desirable to achieve absorption through the highly vascularized oral mucosal tissues in order to avoid first-pass metabolism by the liver.

Many active agents are poorly absorbed in the gastrointestinal tract, even after they are dispersed in the stomach, because of low solubility or slow dissolution rate in the gastric fluids. Tablets may be formulated to be quick dissolving which are commonly placed on the tongue and disintegrate rapidly in the oral cavity. Consequently, they do not overcome a risk associated with choking or gagging that occurs with subjects having limited control of their swallowing reflexes. However, once placed in the mouth, these tablets dissolve rapidly in the saliva to provide a liquid formulation which is then swallowed. However, the disintegration/dispersion time for such tablet dosage forms may take sometimes as long as few minutes. The quick dispersing/dissolving tablets may be formed from a particulate support matrix containing the therapeutic agent, where the particulate support matrix is a protein (U.S. Pat. Nos. 5,807,576, 5,635,210, 5,595,761). Alternatively, the tablet maybe formed from a laminate with several layers and an outer coating (JP 100535518). Tablets have also been manufactured from shearform matrices which are substantially amorphous sugar formed when crystalline sugar is subjected to heat and shear (WO 95/07194; WO 95/35293). Other methods of forming quick dissolving tablets include wet granulation methods (EP 0627 218) and dry granulation methods (EP 0124027A1) and by freeze-drying techniques (EP 0084705A2). Generally, quick dissolving tablets are formed using complex multi-step manufacturing processes. In addition, these tablets may have poor mechanical strength, are fragile and friable and have insufficient holding capacity for active ingredients (U.S. Pat. No. 5,720,974) and may be difficult to store and handle.

Many times therapeutic agents are provided as powders or granules and may be difficult to swallow and cause unpleasant sensations in the mouth. Furthermore, many quick dissolving tablets contain particulates (>25 microns) which leave a “gritty” and unpleasant taste in the mouth. In the older patients, powders may cause choking and discomfort associated with trapping of granules in dentures. Powders and granules are generally packaged in a sealed pouch which requires unsealing before use. This causes problems for geriatric patients and those suffering from arthritis in the fingers, as well as for children. Consequently, problems such as spillage of package contents are common in this group of patients. Furthermore, these oral preparations should be taken with water which for certain patients may be inconvenient and may lead to reduced patient compliance.

Few of the alternatives to solid oral dosage forms addressing aforementioned disadvantages are liquids, syrups, emulsions or suspensions and are considered desirable for pediatric and geriatric patients who have problems in swallowing tablets. However, these dosage forms are often difficult to measure accurately and administer easily without the loss of dose due to spillage. Additionally, liquid formulations deteriorate rapidly upon exposure to heat or atmosphere and consequently have a relatively short shelf life while emulsion/suspension dosage forms may eventually settle and form cakes leading to the loss of dose uniformity. Furthermore, liquid formulations require a relatively large volume and are bulky to store.

In addition to solid and liquid dosage forms, rapidly dissolving buccal/oral delivery systems have been developed using methods including freeze drying. However, the freeze dried preparations are more expensive to manufacture as compared to tablets (U.S. Pat. No. 5,648,093). Furthermore, freeze dried preparations are brittle and fragile when handled and must be kept in dry conditions to avoid disintegration. The instability of freeze-dried preparations has been reduced somewhat by the addition of mannitol (U.S. Pat. No. 4,946,684). WO 9820862 reports a film that is formed according to a method that does not utilize freeze drying and avoids problems described in the art such as rigidity of the films, delayed softening and poor solubility in the mouth (U.S. Pat. No. 4,876,092; EP 0200508; EPO 381194; CA-PS 1-26331; DE 2449865.5; DE 3630603; EP 0452446 and EP 0219762). However, the film described in WO 9820862 relies on the use of at least two different non-ionic surfactants to achieve immediate wettability. In yet another U.S. Pat. No. 4,136,145 issued to Fuchs, a novel film dosage form was disclosed made from water soluble polymers and claimed to be useful for oral, topical or external application. However, this patent failed to address the issues of drug loading and fast dispersion of the films. In the issued U.S. Pat. No. 4,029,758 to Mlodozeniec and Goldberg discloses a method of preparing edible web for film having deposited thereon a medicament. Following the deposition of the medicament another layer of edible film is deposited sealing the medicament. These films upon application to oral cavity release the medicament in controlled fashion. However, this invention does not discuss the fast dispersion/dissolution of the drug and drug loading issues in the fast dispersing films.

A delivery device that addresses the above limitations would represent a desirable improvement on existing delivery systems.

However, aforementioned film formulations and manufacturing methods have resulted in film dosage forms with limited ability to accommodate drug loading beyond small doses and usually in the in the range of 1 microgram to 10 milligrams. However, higher drug loading in the film formulation also results in increased disintegration/dissolution time leading to patient unpalatability. According to present invention where the polymer films are perforated, the drug loading in the film can be enhanced without compromising the fast dispersion/dissolution characteristics of the film dosage forms. As shown in the following examples there are some attempts to present the perforated oral films, however the objective of the prior art is different from the current invention.

### Perforated Film Prior Art:

United States Patent Application 20070071798 by Steve A. Herweck et al. relates to a bio-absorbable stand-alone film that has one or more perforations or depressions provided in the film structure, and the corresponding method of making. The perforations in the bio-absorbable stand-alone film allow the film to expand and conform to a three-dimensional surface of a tissue, even in embodiments where the bio-absorbable film itself is not elastic. The bio-absorbable stand-alone film is generally formed of a naturally occurring oil, or an oil composition formed in part of a naturally occurring oil. In addition, the oil composition can include a therapeutic agent component, such as a drug or other bioactive agent. The perforations in the bio-absorbable film can speed up the biological absorption of the film by allowing body fluids to contact a greater amount of surface area of the film compared to a film without perforations. The bio-absorbable stand-alone film is implantable in a patient for short term or long term applications. As implemented herein, the bio-absorbable stand-alone film is a non-polymeric cross-linked gel derived at least in part from a fatty acid compound. The present invention is clearly for surgical application and not intended for immediate drug release for oral absorption. Additionally the films are based on non-polymeric cross linked gel consisting of fatty acids or oils which are not suitable for oral delivery of drugs since these fatty acids are not soluble in Gastro Intestinal Tract (GIT). The perforations in the film preparation are intended for either holding the drug particles or allowing the body part to protrude through these perforations or allowing the surgical instruments to pass through. Therefore, the perforations referenced in this application are clearly misleading.

United States Patent Application 20070259011 by Sagel et al, is for oral care products comprising a strip and one or more oral care compositions. The oral care composition comprises an oral care active and when applied to the teeth the oral care product delivers one or more oral care benefits.

These oral care systems, preferably for dental care application strips may also be perforated. Perforations can be provided adjacent or below the oral care composition where it is desirable for the oral care active to more readily dissolves or otherwise diffuses into the oral cavity. The provided system is clearly for dental care application and not intended for systemic oral absorption as provided in the form of tablets or capsules or liquid preparations. More importantly this invention does not address the drug loading issues as solved by current invention.

United States Patent Application 20070253990 by Sagel et al. relates to a tooth whitening strip of material having a shape adapted to fit a user's teeth. The strip of material is of a shape so that the strip will substantially cover the front side of at least the user's front four teeth and two canine teeth. The front sides of additional teeth may be covered. The tooth whitening strip will then fold over the tips and onto the back side of one or more of the front four teeth. However, the strip of material will not cover the tips of the two canine teeth. The strip of material may be of any shape that allows for the canines to protrude. Possible shapes include substantially trapezoidal with or without stair stepped sides or notches, and rectangular with notches, stair stepped sides, or recesses.

Any of the sides or edges of the strip may be notched, stair stepped, or arched. By notched it is meant that there is a recess, indentation, or curve of some type. By stair stepped it is meant that the side is not straight and may contain one or more stair steps. The strip may also contain slits, cross-slits, holes, perforations, or any suitable formation that allows for the canines to protrude through or be avoided by the strip of material. In generally, a recess should allow approximately 2 cm for protrusion of the canines. Certainly this invention by Sagel is for tooth whitening and does not address the issues of drug loading solved by current invention.

United States Patent Application 20070237825 by Levy et al., is a polymerized hydrogel composition for hydrating or dehydrating a surface, particularly a dermatological surface, to which it is applied and a method for forming the composition.

One method offered for controlling the rate of release of water to skin surface from the hydrogel composition is by controlling the geometry of the composition itself and, thereby, its exposed surface area. For example, a sheet of the composition with a specified thickness can be designed having perforations of a specific diameter. Diffusion can occur through the surface of the hydrogel and through the surface area created by the edge of each hole. The increase in desorption rate from a perforated sheet compared with a non-perforated sheet can be calculated and predicted. Other geometries that can be produced to control the rate of water release include solid cylinders, hollow cylinders, and hollow cylinders with a non-circular central channel. An example of a cylinder with a non-circular central channel is a cylinder with an internal channel that is “star shaped”. The surface area of the internal “star” can be designed to equal or exceed the surface area of the external surface of the cylinder. The external cross-section of the hydrogel composition can also be non-circular. Examples of non-circular external cross-sections include oval, square, and star-shaped cross-sections. The profile of the hydrogel compositions (internal and external cross-sections) can be designed to meet the required mechanical property criteria and the desired hydrating behavior for specific applications. This invention is clearly intended for dermatological external application only and does not address the delivery of drugs for oral systemic absorption.

In yet another United States Patent Application 20070003495 by Sagel et al., describing a method for whitening teeth are provided. The methods include the steps of providing a strip of material and applying a thin layer of a tooth whitening substance having a whitening active selected from the group consisting of peroxides, metal chlorites, perborates, percarbonates, peroxyacids, hypochlorites, and combinations thereof to a front surface of a plurality of teeth, wherein the amount of the tooth whitening substance is between about 0.05 grams and about 0.4 grams. The method further includes the steps of conforming the strip of material to the front surface of the plurality of teeth and removing the strip of material. The strip may also contain slits, cross-slits, holes, perforations, or any suitable formation that allows for the canines to protrude through or be avoided by the strip of material. In generally, a recess should allow approximately 2 cm for protrusion of the canines. Therefore, the perforations offered into these strips clearly to allow the strips to lodge themselves onto the teeth.

Similar perforations were provided for securing the teeth whitening strips in the U.S. Pat. No. 7,122,199 issued to Sagel et al., however, the invention is not intended for systemic drug delivery of bioactive agents.

In yet another United States Patent Application 20050249782 Perry, Victor et al., is for a drug delivery device having the shape and size of a pocket card and including a drug dispersed in an edible material, is disclosed. The drug delivery device may be conveniently carried by a person, such as in the form of a cash or credit card, allowing easy access to the drug. The person may administer a dosage of the drug by ingesting all or part of the drug delivery device.

As explained in the disclosure, the drug delivery device may comprise one or a plurality of dosages. If drug delivery device contains multiple doses, drug delivery device may be divided into a plurality of segments, each segment may correspond to one dose or part of a dose. Segments may be designed to easily be separated from the remainder of delivery device by breaking the device along the sides of provided segregation perforations. The invention provided with perforations in this application is intended for dose segregation and by no means address the solution solved by current invention.

U.S. Pat. No. 6,936,291 granted to Weibel Michael K disclosed a process for continuous casting of edible cellulose-containing film sheets having unique physical characteristics. The cellulose film sheets have diverse applications in the processed food industry.

The film sheets of the invention can be beneficially incorporated into a variety of comestible substances, and in particular, frozen or par-baked dough pieces, to which the film sheets are readily laminated. For example, the film sheets of the invention are useful as an edible baking substrate for frozen pizza products, as they have sufficient structural stability to resist the deformation of the pizza dough over the rods of an oven rack, which can result in uneven baking. The resulting film sheet can be formed into various shapes, e.g. by die-cutting or the like, depending on the intended use. The film sheets may also be perforated, if desired. Therefore, the perforations in the invention are only to segregate the pieces for easy handling but not provided to address the fast dispersion/dissolution of the films.

In yet another United States Patent 20050186253 by Lee et al., discloses a method of manufacturing a bioactive fluid dose on an ingestible sheet, comprising the steps of advancing the ingestible sheet to a dispense position, and activating a fluid ejector to dispense essentially a drop of a bioactive fluid onto the ingestible sheet.

The ingestible sheet in this invention is in the form of a roll that contains perforations that delineates each dosage form. Preferably, after the bioactive fluid is dispensed on the dosage form the user or system separates the dosage form by tearing, by putting along the perforations, or by punching out the dispensed areas of the sheet. Therefore, this invention does not address the problem of drug loading and fast dispersion/dissolution solved by current invention.

The above mentioned patent applications and patents refer to the use of perforations in the edible polymer films in a non specific fashion. The perforations in most of the references in these patents are provided to either segregate the dose or a means to separate the individual doses by tearing along the sides of the perforations. In yet another invention these perforations are provided to secure the films in place. One patent addresses the faster dissolution of applied hydrogel as wound dressing but it is for only external application and treatment of wounds on dermis. Therefore the use of perorations in the films/strips is just a convention and conveniently misleading.

### SUMMARY OF INVENTION

It is known to the pharmaceutical sciences that water soluble polymer based films can be used to deliver the drug substance to oral cavity. However, the thickness of such films needs to be substantially, thin in order to achieve rapid dissolution on the tongue upon application on the tongue. This requirement significantly limits the drug loading capacity of the films restricting the application of the technology to only low dose drug delivery. Additionally, any increase in thickness of the film results in increased dissolution/dispersion times. These disadvantages render these delivery systems as less palatable to patients.

A primary objective of the present invention is to provide a oral fast dispersible/dissolvable edible polymer based perforated film/strip dosage form that will overcome the shortcomings of the prior art and provide a system that will disperse/dissolve faster even when the thickness of the strip is increased due to increased drug loading.

The current invention particularly addresses the issues posed by earlier inventions such as fast dispersion/dissolution and drug loading limitations. Therefore, allowing these delivery systems to be flexible to accommodate higher drug loads without compromising the fast dispersion/dissolution characteristics. Additionally, this invention provides much faster dispersion/dissolution to the system even at low drug load. The current invention also enables the use of polymers that would otherwise have not been used due to lower water solubility.

Accordingly, an objective of the current invention is to provide, microperforated or perforated water soluble polymer based edible films for the oral delivery of drugs.

Another objective of the invention is to provide a means for the faster dosage form dissolution or dispersion after dosage form administration.

Another objective of the invention is to provide a means to accommodate higher drug loads into the delivery system without compromising the dispersion/dissolution rates.

Another objective of the invention is to improve the ability of the strip/film to accommodate higher drug loading while preserving fast dispersion/dissolution characteristics.

Another objective of the invention is to improve the ability of the strip/film dosage form to disperse/dissolve faster even for low drug loaded thinner films. Additionally, present invention enables the usage of those polymers otherwise may not allow the fast dispersion/dissolution of the dosage form.

Another objective of the current invention is to allow delivery of drugs to oral cavity at various dose levels. Provide the dosage form with lower/higher doses while preserving fast dispersion/dissolution rates.

Another objective of the invention is to provide increased dissolution surface area to the delivery system for the enhanced dissolution/dispersion.

Another objective of the invention is to provide flexible usage of polymers, which could otherwise not be suitable for the preparation of dosage form.

Another objective of the invention is to provide means to incorporate low water soluble drugs into these dosage forms for effective delivery.

The general purpose of the present invention is to provide a new oral drug delivery system that has several advantages over other forms and novel features that result in new oral drug delivery system which is not anticipated, rendered obvious or even implied by any of the prior art of oral cavity delivery system.

To attain this, the present invention generally consists of polymer, plasticizer, buffer agent, disintegrants, effervescence agents, sweeteners and drug formed into a flexible film.

Generally the polymers include either water soluble polymers or pH dependent polymers or thermoplastic polymers or combination thereof along with suitable amounts of water soluble/water miscible/water insoluble plasticizers, buffering agents, disintegrants, effervescence agents, flavorants, sweeteners, emulsifying agents and suitable amounts of drug substance. The drug substance can be from any class of drugs either acidic or basic or zwitterionic. The dose range of these drugs can be from few micro grams to few hundred milligrams. The resultant combination of these polymers, plasticizers, buffering agents, disintegrants, effervescence agents, flavorants, sweeteners and drugs can be a solution, suspension or emulsion which can be spread to form films upon drying.

These systems can be made either in the presence of water or any suitable organic solvent or combination of both.

Such prepared films usually contain drug levels in higher percentages or at same percentage as present commonly known films on the market. Additionally, these films can be dissolved or dispersed in oral cavity at faster rates than traditional films due to the presence of perforations throughout the body of the strip.

The perforations can be made in the body of the film by several known methods such use of pins/punches or laser drills or mechanical drills or conventional methods. The holes can be of any shape and size, however, the film upon perforations still provide specific amounts of drug when ingested.

These and other details of invention will be described in connection with the accompanying drawings, which are furnished only by the way of illustration and not in limitation of the invention, and in which drawings:

### BRIEF DESCRIPTION OF FIGURES

FIG. 1 shows the current invention in its simplest embodiment. (1) is polymer fast dispersing/dissolving film and (2) are the perforations in the film according to current invention.

FIG. 2 schematically shows the preparation of the dosage form. (1) is polymer film roll acting as backing matrix supporting the polymer film forming material; (2) is a steam jacketed mixer/blender/homogenizer where the formulation composition for film forming material is mixed and the delivered onto the backing film at a constant rate under constant stirring; (3) is a spreader, in this example it is a doctor knife assembly, spreads the film forming material delivered to the backing film evenly and to a predetermined thickness; (4) are hot air ovens, where the hot air is sprayed onto the wet coated surface through air nozzles (5) or battery of nozzles or circularly moving nozzles or other type to remove the solvent/moisture from the film forming matrix at constant precalculated rates; the drying of the material can be accomplished by various means including hot air, laser energy, irradiation energy or IR lamps, heating coils and other mechanisms; (6) is the up wound coated material roll rolled up with additional barrier layer film/membrane used to prevent two coated layers to come in contact with each other; (7) is the perforator or such mechanism which can form perforations into the coated membrane. Alternatively the perforations are formed at later stage prior to packaging as shown in FIG. 3.

FIG. 3 presents the perforation formation and packaging process schematically; (1) is coated film roll passing under (2) perforator or such perforation formation mechanism to result in a (3) perforated film. Both backing and separation membranes are removed from the coated film prior to its insertion under the perforator (2); the (3) perforated film is then slit into appropriate dimension and (4) individually packaged; the strips can also be packaged as (5) blister pack which then carried in the pocket or alternatively can be packed into (6) canisters after individually packed.

### DETAILED DESCRIPTION OF THE INVENTION

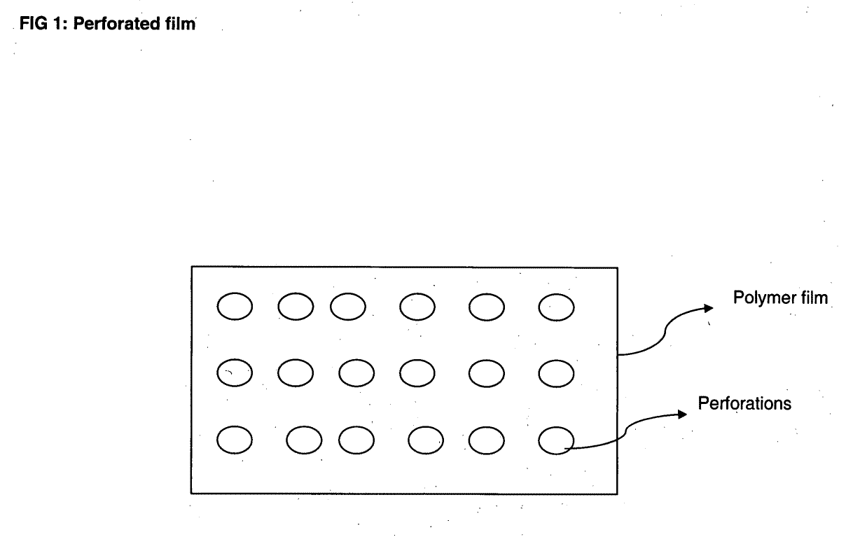

A novel dosage oral fast dispersible/dissolvable dosage form and the manufacturing method, and finally the application of it in human subjects is provided. In its simplest embodiment (FIG. 1), the dosage form comprises of a water-soluble or pH dependent or thermoplastic polymer, plasticizer, disintegrant/s, effervescence agents, sweetener, suitable flavors with perforations on the film body including appropriate pharmacologically effective amount of bioactive agent.

In an embodiment of the invention, the polymer material used in the manufacture of the film includes a polymer selected from the group consisting of a natural, semi-natural and synthetic biopolymer such as a polysaccharide and a polypeptide. In addition to the film forming polymer material, the film may comprise of one or more of an solubiling agents, suspending agent, an emulsifier, a plasticizer, a taste modifying flavorant, a water soluble/dispersible/suspendable inert filler, a preservative, a buffering agent, a combination of effervescent agents, disintegrants, a coloring agent, a permeation enhancer, an anti oxidant, a chelating agent and a stabilizer. The film may further include an active agent selected from various pharmacologically active agents either a therapeutic agent, a dietary supplement and/or other personal hygiene agents.

Embodiments of the current invention may comprise bioactive agents in the therapeutically effective levels of sildenafil citrate, Vardafil, Tedalafil citrate, loratidine or estradiol, progesterone or any other bioactive agents in the film dosage form. The active agents can be incorporated into films by various pharmaceutical formulation processes including molecular dispersion, solubilization, suspension, emulsification or other methods depending on the physicochemical properties of the bioactive material. One or more bioactive agents can be incorporated in the film either in one film or in bilayer or trilayer film in the concentration range of 0 to 90%. In an embodiment of the invention, the solubilizer may be in the concentration range of 0 to 25%, emulsifier in the range of 0.1-10% weight, suspending agent in the range of 0 to 20%. The water soluble fillers may include a concentration range of 0-75%, flavorants in the range of 0-2%, sweeteners in the range of 0-10% and the preservative may be in the range of 0 to 10%.

In the embodiment of invention, the film comprises perforations. The perforations may be of any size and shape and in any number in the film body. The perforations can be in various directions and designs. The perforations are made into films by various mechanisms including mechanical drilling/punching, burning with pins into films, laser burning or formed during the layering of the film forming material on to a protruded base film surface which is finally peeled to provide holes in the films.

In the embodiment of invention, the perforations are made into films of various thicknesses. The thickness of the film forms basis for the number of perforations in the film leading to faster dispersion/dissolution of the films in the oral cavity/body cavity/body surface of any subject including humans and animals.

In embodiments of the invention, the dosage unit may further include any of the following features: a dry film thickness in the range of 1-50 mil, more particularly less than 20 mils, a tensile strength greater than 500 psi, a modulus in the range of 15,000-200,000 psi, a tear propagation resistance in the range 0.0001N-1N, a disintegration time in a range from 1-600 seconds, a dissolution time in a range from 10-1000 seconds, and a percentage elongation less than 20%.

In embodiments of the invention, manufacturing process for the films are provided. The typical process includes dissolution/dispersion of the film forming material either in water or a suitable solvent or a mixture thereof. To the polymer solution mix bioactive agent which is previously dissolved/solubilized/suspended/emulsified/otherwise dispersed by known pharmaceutical practices is added under agitation to result in homogenous mixture. Alternatively, the bioactive agent may also be dissolved/solubilized/suspended/emulsified/otherwise dispersed by known pharmaceutical practices directly into the polymer/polymer solution. Also, at least one reagent selected from the group consisting of an emulsifier, a plasticizer, a taste modifier, a water soluble inert filler, a coloring agent, a preservative, a permeation enhancer, a stabilizer, effervescent agent, disintegrant and a buffering agent to form a coatable mixture. The method may further include the step of coating the mixture onto a backing film. In a further embodiment, the reagents including: a hydrocolloid, an active agent, and at least one reagent selected from the group consisting of an emulsifier, a plasticizer, a taste modifier, a water soluble inert filler, a coloring agent, a preservative, a permeation enhancer, a stabilizer, and a buffering agent, may be combined in any order in a vessel equipped with heating mechanism such as steam jacket and a mechanical mixing device. The resultant mixture is then applied onto a backing film using a known coating mechanisms such as doctor knife or reverse roll coating etc. The coated backing film then passed through a drying chamber assembly where the heated air at predetermined temperatures is blown on to the film to remove the water/solvent/combination thereof at a predetermined rate from the film.

The perforations into the film are formed by one of the methods including passing the exiting coated film through as roller punching mechanism/drilling mechanism. The perforations are formed into the film by laser drilling the holes upon exit of the film from the drying chamber. The perforations are also formed by bringing the film surface in contact with hot pins. Alternatively, the holes/perforations can be formed in the film by any of the aforementioned methods or other methods prior to packaging but soon after peeling it from the backing film. Yet another method of forming the perforations is by means of forming a matrix of the film while coating on to the backing film or by effervesce carbon dioxide evolution during coating of the film. Nonetheless, as can be perceived by a skilled pharmaceutical formulation experts that other perforations formation methods not mentioned in this invention should not be considered as limitations of the invention. Following perforations, the film is then cut into appropriate dimensions accommodating correct dose strength of the bioactive agent and either packaged individually or stacked in a dispensing container.

The dosage form in the invention may be used as a vehicle for delivering a wide range of active agents to GIT or oral cavity. For example, the active agent may be a small molecule, a peptide, a protein, a nucleic acid including antisense molecules or other biological or synthetic molecules.

Embodiments of the present invention include a process, composition and method of use for a fast dispersing/dissolving film for local and systemic delivery of pharmaceutical agents including to the oral cavity, the systemic or local delivery of an active agent and skin surface, vaginal, rectal, and ocular surfaces. The films can also be applied to periodontal, lingual, sub-lingual, buccal, gingival, and palatal surfaces.

The present invention also is applicable for vaginal delivery of such agents as contraceptive agents including nonoxynol or anti-infectives including antifungal agents, antibacterial agents, anti-viral agents, fragrance or hygiene agents.

The embodiments of the invention provide improved film dosage forms to deliver bioactive agents that are recommended for all age groups and that physician, parents, patients and family members can administer easily. These dosage forms are economical to prepare and have an extended shelf life. They are easy to handle and non-tacky before administration so as to avoid disintegration prior to use and are conveniently packaged for shelf life, ease of storage and distribution. The dosage form may be administered to the subject by placing the film on body cavity or surface.

Embodiments of the invention provide a delivery system for bioactive agents and other active agents that will fast disperse/dissolve and completely release their contents on the applied surface such as tongue. The release of the active agent occurs without mastication or the need for intake of water. However, the dosage form can be administered with aid of water if needed. Furthermore, embodiments of the invention further provide improvements that include: improved organoleptic properties (smell and taste), and texture and feel of dosage forms intended to be placed in the oral cavity; a dosage form which “melts” in the mouth and leaves a smooth pleasant after feel following dissolution; Depending on the optimal program for a specific application of the invention, the disintegration time and the dissolution time can be controlled within a prescribed range by adjustment of the formulation and the thickness of the film. In some cases, it is desirable for release of the active agent to occur after dissolution of the film. For these applications, the active agent may be encapsulated in a material with dissolution properties that are different from those of the hydrocolloid. Encapsulation of the active agent also may be utilized to achieve masking of taste for active agents that are bitter. In some cases, two or more different active agents may be included in the film. An example where multiple active agents frequently are administered is cold medications, which often contain several active agents.

“Coating mixture” is defined here and in the claims as a viscous and homogeneous mixture of film forming polymers, active agents and other additives in water or a solvent or a mixture thereof. The coating solution is treated according to the method of the invention to form a film.

“Subject” is defined here and in the claims as a human or animal species.

“Thickness” is defined here and in the claims by measurements in mil (a mil=one thousandth of an inch) determined using appropriate thickness gauges.

Flavoring agents include the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, chamomile, sugar, dextrose, lactose, mannitol, sucrose, xylitol, malitol, acesulfame potassium, talin, glycyrrhizin, sucralose, aspartame, saccharin, sodium saccharin, sodium cyclamate and honey.

Emulsifying agents include polyvinyl alcohol, sorbitan esters, cyclodextrins, benzyl benzoate, glyceryl monostearate, polyoxyethylene alkyl ethers, polyoxyethylene stearates, poloxamer, polyoxyethylene castor oil derivatives, hydrogenated vegetable oils, bile salts, polysorbates and ethanol. Plasticizers may include glycerin, sorbitol, propylene glycol, polyethylene glycol, triacetin, triethyl citrate (TEC), acetyl triethyl citrate (ATEC) and other citrate esters.

Bioactive agents for both human and animal ailment treatment include therapeutic agents, nutritional supplements and hygiene aids from the pharmacological categories of analgesics, alpha and beta adrenergic receptor blockers, anti-Alzheimer's disease medication, antianginal, antianxiety, antiarrythmics, antiarthritics, antibiotics, anticoagulants/thrombolytics-, anticonvulsants/anti-Parkinson medication, anti-depressants, anti-diabetics, anti-diarrheal, anti-epileptics, anti-fungal, anti-gout, anti-heartworm medication for dogs, anti-histamines, anti-hypertensives, anti-inflammatories, anti-infectives, antimigraines, anti-nasuants/antiemetics, anti-neoplastics/anti-tumor active agents, anti-pruitics, anti-psychotics, anti-pyretics, anti-spasmodics, anti-virals, bronchial dilators/anti-asthmatics, calcium antagonists, cardiac agents, cardiotonics, central nervous system actives, contraceptives, coronary vasodilators, cough/cold remedies, dietary supplements, including vitamins and minerals, diuretics, erectile dysfunction agents, fertility active agents, flea control agents for animals (Ivermectin), HZ receptor antagonists, herbal actives, hormones, hypoglycemics, hypolipidemics, muscle relaxants, ovulation stimulators, peptide active agents, polypeptide active agents, proteins such as insulin, calcitonin, LHRH, cholesterol lowering agents, sedatives and hypnotics, erectile dysfunction agents, sleep aids, smoking cessation aids, steroids and steroidals, tranquilizers, laxatives, ophthalmic preparations, nutritional supplements, breath fresheners, breath deodorants, saliva substitutes, antigingivitis agents, anti-cavity agents, anti-plaque agents, diagnostic indicators, and local anesthetics. Also included are active agents for treatment of osteoporosis, hormone replacement, treatment of periodontal disease, antiseptics, corticosteroids, non steroidal anti inflammatory agents, antiviral agents and vaccines.

Water soluble inert fillers include mannitol, xylitol, sucrose, lactose, maltodextrin, dextran, dextrin, modified starches, dextrose, sorbitol, and dextrates.

Effervescence and buffering agents include acidulants and alkalizing agents including citric acid, fumaric acid, lactic acid, tartaric acid, malic acid, as well as sodium citrate, sodium bicarbonate and carbonate, sodium or potassium phosphate and magnesium oxide.

Coloring agents may include FD & C coloring agents, natural coloring agents, and natural juice concentrates, pigments and opacifying agents such as titanium oxide, silicon dioxide and zinc oxide.

Stabilizers and preservatives may include anti-oxidants, chelating agents, and enzyme inhibitors such as ascorbic acid, vitamin E, butylated hyroxyanisole (BHA), butylated hydroxytoluene (BHT), propyl gallate, dilauryl thiodipropionate, thiodipropionic acid, gum guaiac, citric acid, edetic acid and its salts, glutathione, anti-microbial agents sodium benzoate, parabens and derivatives, sorbic acid and its salts, propionic acids and its salts, sulfur dioxide and sulfites, acetic acid and acetates, nitrites and nitrates and others.

In embodiments of the invention, a film former concentration can be in the range of 5-99% of the dry weight of the films, more particularly greater than 5-10%. The low dry tack properties of the film provide for a physically attractive and easily handled film that is neither fragile nor sticky and can be easily removed from packaging and placed in the oral cavity or skin or other mucosal surfaces. These properties render the films suitable for easy making, packaging, handling and application.

In the embodiment of invention, the film forming polymer can be a hydrocolloid, a water soluble polymer film forming material which are pharmaceutically acceptable polymers from the following categories including synthetic and natural polymer material. These can include polyanionic, polycationic and uncharged polymer species including Cellulose polymers (synthetic) such as Hydroxypropylmethyl cellulose (HPMC), Hydroxy propyl cellulose (HPC), Methyl Cellulose (MC), Carboxymethyl cellulose (CMC), Starches, and Cellulose polymers (natural) such as Acacia, Tragakanth, Carrageenan, Pullulan and Other water soluble polymers including Polystyrene sulfonates, Polyethylene oxides/Polyethylene glycols, Polyacrylic acids, Polybenzenesulfonic acids, Polyethylenimine, Poly diallyldimethyl ammonium chloride, Polyallylamine hydrochloride, Polyvinyl pyrrolidone (PVP) and Gelatin The water soluble polymeric materials also can be pectin and derivatives, guar gum, xanthan gum, gellan sodium salt, propyleneglycol alginate, starches (amylose, amylopectin), modified starches, hydroxyethyl starch, pullulan, carboxymethyl starch, gum ghatti, okra gum, karaya gum, dextrans, dextrins and maltodextrins, konjac, acemannan from aloe, locust bean gum, tara gum, quince seed gum, fenugreek seed gum, scleroglucan, gum arabic, psyllium seed gum, tamarind gum, oat gum, quince seed gum, carrageenans, scleraglucan, succinoglucan, larch arabinogalactan, flaxseed gum, chondroitin sulfates, hyaluronic acid, curdlan, chitosan, deacetylated konjac, and rhizobium gum.

Additionally, in embodiments of the invention, the water soluble hydrocolloid may be a polypeptide or protein exemplified by gelatins, albumins, milk proteins, soy protein, and whey proteins. The hydrocolloid may further be selected from a group of synthetic hydrocolloids exemplified by any of the following: polyethylene-imine, hydroxyethyl cellulose, sodium carboxymethyl cellulose, carboxymethyl cellulose, hydroxypropyl cellulose, hydroxypropyl methyl cellulose, methyl cellulose, ethyl cellulose, polyacrylic acids, low molecular weight polyacrylamides and their sodium salts (carbomers), polyvinylpyrrolidone, polyethylene glycols, polyethylene oxides, polyvinyl alcohols, pluronics, tetronics, and other block co-polymers, carboxyvinyl polymers, and colloidal silicon dioxide.

In the embodiment of invention the film forming material may also be a water insoluble polymer film forming material which are pharmaceutically acceptable polymers from the following categories including synthetic and natural polymer material such as Acrylic/methacrylic acid copolymers including pH dependent polymers such as acrylic acid/methacrylic acid copolymers (eg: Eudragit L and S polymers); pH independent polymers including acrylic/methacrylate copolymers (eg: Eudragit RL and RS polymers), and Carbomer polymers (eg: Carbopols 931 and 974 polymers and others).

Using above polymers by themselves or in combination with other polymers and suitable amounts of plasticizer content the films can be made using a Braive laboratory bar coater or a BASF lab coater or Elcometer or other industrial film coating systems such Mathis AG coating system onto a polyester sheet and dried at suitable temperature to a suitable relative humidity and suitable thickness which is then perforated using methods such as drilling, burning, punching, laser drilling and even by matrix forming. Methods for manufacturing the film dosage form of the invention include the solvent casting methods or alternatively extrusion methods involving blending ingredients to form a film using mechanical force and moderate heat. A preferred embodiment of the invention utilizes a hydroxypropyl methyl cellulose having a methoxy content of about 19-30% and hydroxypropyl content of 7-12% and a molecular weight of approximately 50,000-250,000 daltons.

In addition to hydrocolloids and the active agents, the films may contain any or all of the following ingredients: emulsifying agents, solubilizing agents, wetting agents, suspending agents, taste modifying agents, plasticizers, active agents, water soluble inert fillers, preservatives, buffering agents, effervescence agents, coloring agents, and stabilizers. In a preferred embodiment, the percentage dry weight concentration of at least single ingredients incorporated in a film in each of the following categories is as follows: emulsifying agent (0.1% 10%), plasticizer (0.5-20%), active agents (0.01-80%), taste modifying agents (0.1-10%), coloring agents (0.01-5%), water soluble inert fillers (0.5-50%), preservatives (0.01-10%), buffering agents (0.1-10%) and stabilizers (0.01-5%).

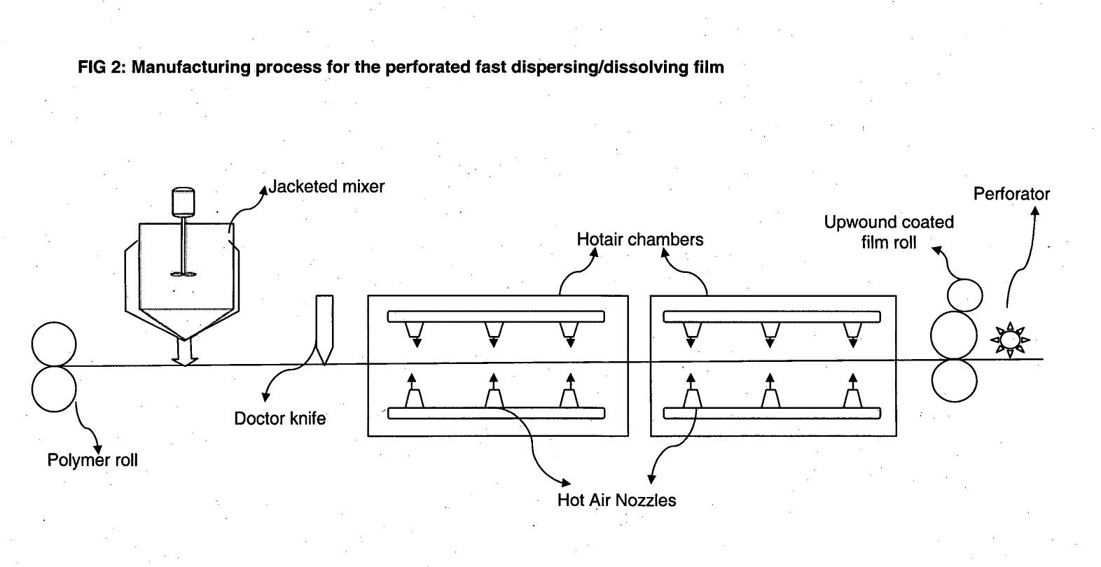

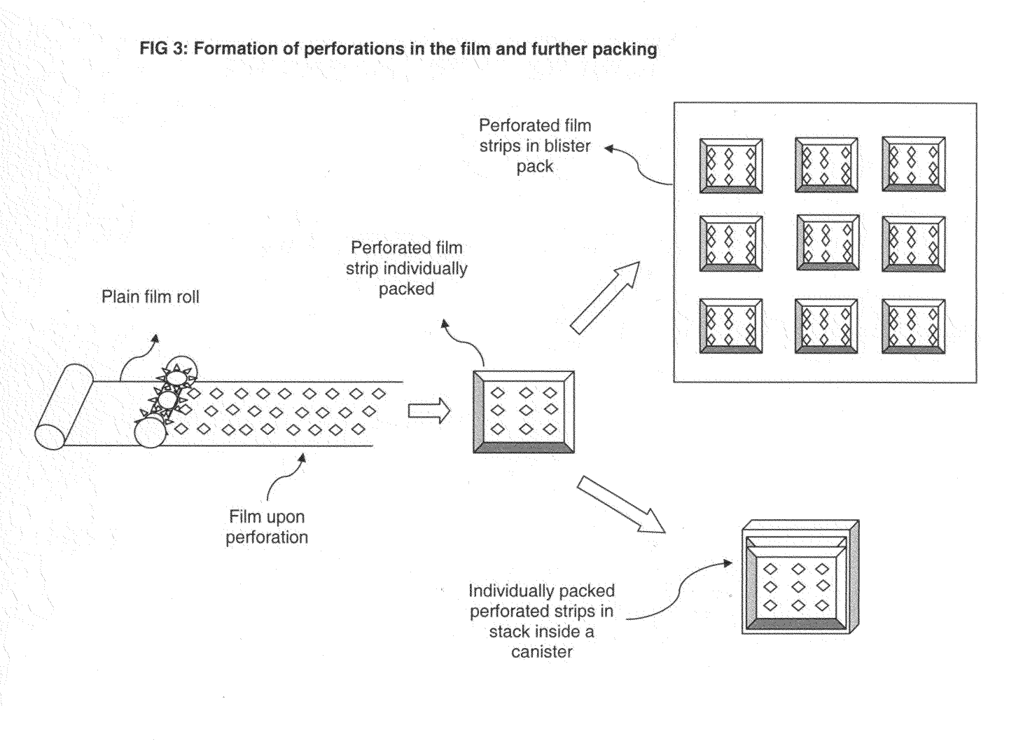

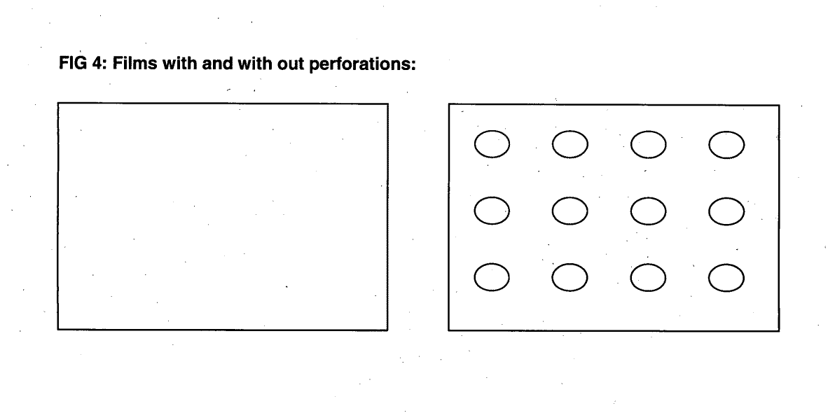

In an embodiment of the invention, the film forming polymer is dissolved in water or hydroalcoholic mixture. The bioactive agent is either dissolved or dispersed in water or hydroalcoholic mixture and added to the polymer solution under mild agitation. In addition to the active agent and the film forming polymer, any of the ingredients listed above may be added and dispersed or dissolved uniformly in to hydrocolloid solution. The volatile active ingredients and flavoring agents can be incorporated before or after film forming. This homogeneous mixture (coating solution) with a solid content of 5-80% and a viscosity of 300-25000 cps is then either left over night to remove air bubbles or degassed under vacuum. This coating material is then coated on to the non-siliconized side of a polyester film at 5-50 mil or 0.01 mm to 5 mm wet film thickness, more preferably 10 microns to 2000 micron wet film thickness and dried in an hot air oven at 40-200 C. The manufacturing process for forming the dosage unit is illustrated in FIG. 2. The dry film formed by this process is a glossy, stand alone, self supporting, non-tacky and flexible film. The dry film is then perforated and cut into a suitable shape and surface area for administration. The perforation formation and packaging of the final films is illustrated in FIG. 3. The size of the film varies based on the dose to be delivered. Unlike other conventional dosage forms, the film dose form therefore provides the flexibility of accommodating dose at ease by changing the dimensions of the film. The sized films are then packaged into single unit packages, multi-unit packages including blisters and dispensers.

Presented examples in the invention are mere illustrations of reducing the invention to practice but not limited to these illustrations. Other means and methods to achieve films with perforations are possible to the skilled specialist therefore, should not be considered as limitations of the invention.

Various examples provided here show that how the properties of the films vary when different type or grade of film forming polymer and other components such as plasticizers, emulsifiers, suspending agents, flavorants, colorants, stabilizers and others. According to the invention these variables in film characteristics can be utilized to the advantage of fast dispersion/dissolution of the film by incorporating perforations on the film surface.

### EXAMPLES

In the embodiment of invention, the fast dispersing/dissolving perforated films are prepared using formulation compositions presented in Table 1.

The polymer based films are prepared as explained in the following examples using formulation composition presented in Table 1. The prepared polymer composition along with other ingredients including plasticizers, fillers, taste masking agents, disintegrants, colorants is then cast on to a backing membrane either using a simple draw down blade or coated onto the moving roll of backing membrane and dried either in an oven or in the dynamic heating chamber.

The prepared films are then drilled or punched or laser drilled or burnt to form perforations in the body of the film. The prepared films resulted in thickness range of 50 to 10000 microns based on the drug loading levels. Drugs from various pharmacological categories intended for systemic absorption or local action or cosmetic purposes can be loaded into these perforated films. In the following examples, two drugs, Sildenafil citrate and Loratidine, are used in the preparation of perforated fast dispersing/dissolving film dosage forms.

The following examples are set forth to further illustrate the oral films and methods of preparation. The below examples, however, should not be construed as limitations to the present invention in any manner.

TABLE 1
Formulation compositions of fast dispersing/dissolving films

| S. No | Ingredient | Example-1 | Example-2 | Example-3 | Example-4 | Example-5 | Example-6 | Example-7 | Example-8 | Example-9 |
|---|---|---|---|---|---|---|---|---|---|---|
| 1 | Methocel E | 0.800 g | 0.300 g | 0.300 g | — | — | 0.400 g | 0.400 g | — | — |
| | 15 | | | | | | | | | |
| 2 | Methocel E4 M | — | 0.100 g | 0.100 g | 0.100 g | 1.000 g | 0.150 g | 0.150 g | — | 0.100 g |
| 3 | Methocel E 5 | — | — | — | 1.000 g | — | — | — | 0.700 g | 0.800 g |
| 4 | Plasdone K | 0.100 g | 0.150 g | 0.150 g | — | — | — | — | — | — |
| | 29-32 | | | | | | | | | |
| 5 | Polyplasdone | 0.100 g | — | — | — | — | — | — | — | 0.150 g |
| | XL-10 | | | | | | | | | |
| 6 | Sildenafil | 1.000 g | 0.100 g | 0.150 g | 0.100 g | 0.300 g | 0.300 g | 0.300 g | 0.300 g | 0.300 g |
| | Citrate | | | | | | | | | |
| 7 | HPBCD | 2.300 g | — | — | — | — | — | — | — | — |
| 8 | Maltodextrin | — | 1.500 g | 1.500 g | — | — | — | — | — | — |
| | (M 180) | | | | | | | | | |
| 9 | Instant | — | — | — | 0.800 g | 1.000 g | — | — | 0.300 g | 0.100 g |
| | purecote B | | | | | | | | | |
| | 793 | | | | | | | | | |
| 10 | Propylene | 0.060 g | 0.050 g | 0.050 g | 0.400 g | 0.400 g | — | — | 0.200 g | — |
| | glycol | | | | | | | | | |
| 11 | Glycerol | 0.060 g | 0.050 g | 0.050 g | — | — | 0.200 g | 0.200 g | — | 0.200 g |
| 12 | Sorbitol (70% | 0.2 ml | 0.15 ml | 0.200 g | — | — | — | — | — | — |
| | solution) | | | | | | | | | |
| 13 | Triethyl | — | — | — | — | — | — | — | — | — |
| | citrate | | | | | | | | | |
| 14 | Sucralose | 0.500 g | 0.200 g | 0.200 g | — | — | 0.100 g | 0.100 g | — | 0.050 g |
| 15 | Tween 80 | 0.1 ml | 0.10 ml | — | — | — | — | — | — | — |
| 16 | Menthol | 0.100 g | — | — | — | — | — | — | — | 0.050 g |
| 17 | MCC | — | — | — | — | — | — | — | — | 0.050 g |
| 18 | Water | 8.00 ml | 10.00 ml | 10.00 ml | 20.00 ml | 15.00 ml | 10.00 ml | 10.00 ml | — | 9.60 ml |
| 19 | Ethanol | 12.00 ml | — | — | — | — | — | 0.40 ml | 15.00 ml | 3.40 ml |

### Formulation and Formation Procedure:

In its simplest embodiment of the invention, the following explained general film preparation method is followed. However, it is commonly understood for a skilled formulation expert that the current invention can be reduced to practice using various other manufacturing methods.

Preparation 1: In general the film forming polymers are either dissolved or dispersed in suitable solvent and degassed using either vacuum method or other suitable methods. In one example the following composition is used.

| | |
|---|---|
| Methocel E 5 | 6.40 g |
| Methocel E 4M | 0.80 g |
| Water | 56.0 g |

Procedure: Methocel E5 and E4 M were weighed in a beaker and wetted with 40 ml of hot water (80° C.) and then 16 ml hot water was added, mixed well and left over night or degassed under vacuum.

### Preparation 2:

| | |
|---|---|
| Purecote B 793 | 0.80 g |
| Sucralose | 0.40 g |
| Coffee Flavor | 1.32 g |
| Water | 32.0 g |

Procedure: All the three ingredients weighed separately and mixed in a beaker and then added 10 ml of hot water (80° C.) and mixed well.

### Preparation 3

| | |
|---|---|
| Sildenafil Citrate (bioactive agent) | 2.4 g |
| Glycerol | 2.4 ml |

Procedure:
1. In a beaker Sildenafil Citrate is thoroughly wetted with glycerol and left aside for 15 mins
2. To the above beaker, weighed quantities of preparation 1 is added, mixed well and then preparation 2 is added. After mixing, it is either kept aside for 2 hours or degassed further to remove any froth from the preparation.
3. The mixture is then cast on the backing membrane or liner and oven dried at 60° C.
4. Alternatively this mixture is also spread on the moving roll of the polyester film using a doctor knife followed by drying under controlled temperature at various drying rates in a serial hot air dryer.
5. Following drying the films are detached from the backing membrane and perforations were made by punching holes using Dremel drill machine and bits. Alternatively the perforations are punched with a hollow tube.
6. Such prepared perforated films are then tested in-vivo by placing the appropriately cut film on the center of tongue. Gentle motion of the tongue was provided by slightly moving the film against the upper palate of the oral cavity. The time of complete dispersion/dissolution was noted for the films with and without holes.

### Example 1

0.8 g of Methocel E15, 0.1 g of Plasdone K29/300, 0.1 g of Polyplasdone XL10, 1.0 g of Sildenafil Citrate, 2.3 g of HPBCD (hydroxylpropyl P cyclodextrin), 0.06 g of Glycerol, 0.06 g of Propylene Glycol, 0.2 ml of 70% Sorbitol solution, 0.5 g of Sucralose, 0.1 ml of Tween 80, 0.1 g of Menthol, 8 ml of water and 12 ml of Ethanol were added in the fashion presented in the preparation procedure followed by drying and perforating the resultant film. The formed films were uniform in appearance and able to dissolve rapidly.

### Example 2

0.3 g of Methocel E15, 0.1 g of Methocel E4M, 0.15 g of Plasdone K29/300, 0.1 g of Sildenafil Citrate, 1.5 g of Maltodextrin (M180), 0.05 g of Glycerol, 0.05 g of Propylene Glycol, 0.15 ml of 70% Sorbitol solution, 0.2 g of Sucralose, 0.1 ml of Tween 80, 10 ml of water were added in the fashion presented in the preparation procedure followed by drying and perforating the resultant film. Good films were obtained with slight rough surface which dissolved instantly in the mouth. However, the drug loading in these films was 0.8 mg/sq.cm.

### Example 3

0.3 g of Methocel E15, 0.1 g of Methocel E4M, 0.15 g of Plasdone K29/300, 0.1 g of Sildenafil Citrate, 1.5 g of Maltodextrin (M180), 0.05 g of Glycerol, 0.05 g of Propylene Glycol, 0.15 ml of 70% Sorbitol solution, 0.2 g of Sucralose and 10 ml of water were added in the fashion presented in the preparation procedure followed by drying and perforating the resultant film. In this formulation the surfactant Tween 80 was removed to obtain better films. Strong and slightly brittle dissolvable films which dissolved slightly slower than previous films in the mouth.

### Example 4

1.0 g of Methocel E5, 0.1 g of Methocel E4M, 0.8 g of Instant Purecote B793, 0.1 g of Sildenafil Citrate, 0.4 g of Propylene Glycol and 20 ml of water were added in the fashion presented in the preparation procedure followed by drying and perforating the resultant film. The formed films were uniform, clear and transparent in appearance and able to dissolve rapidly. However, the drug loading was comparatively less in this formulation.

### Example 5

1.0 g of Methocel E5, 1.0 g of Instant Purecote B793, 0.3 g of Sildenafil Citrate, 0.4 g of Propylene Glycol and 15 ml of water were added in the fashion presented in the preparation procedure followed by drying and perforating the resultant film. The formed films were at higher drug loading, uniform, clear and transparent in appearance and able to dissolve rapidly.

### Example 6

0.4 g of Methocel E15, 0.1 g of Methocel E4M, 0.3 g of Sildenafil Citrate, 0.2 g of Glycerol, 0.1 g of Sucralose, and 10 ml of water were added in the fashion presented in the preparation procedure followed by drying and perforating the resultant film. Good films were obtained with good mouth feel.

### Example 7

0.4 g of Methocel E15, 0.15 g of Methocel E4M, 0.3 g of Sildenafil Citrate, 0.2 g of Glycerol, 0.1 g of Sucralose, 10 ml of water and 0.4 ml of ethanol were added in the fashion presented in the preparation procedure followed by drying and perforating the resultant film. Good films were obtained with good mouth feel.

### Example 8

0.7 g of Methocel E5, 0.3 g of Instant Purecote B793, 0.3 g of Sildenafil Citrate, 0.2 g of Propylene Glycol and 15 ml of water were added in the fashion presented in the preparation procedure followed by drying and perforating the resultant film. The formed films were at higher drug loading, uniform, clear, transparent and thick in appearance and able to dissolve rapidly over the unperforated films.

### Example 9

0.8 g of Methocel E5, 0.1 g of Methocel E4M, 0.15 g of Polyplasdone XL-10, 0.1 g of Instant Purecote B793, 0.3 g of Sildenafil Citrate, 0.2 g of Glycerol, 0.05 g of Menthol, 0.05 g of Microcrystalline cellulose, 9.6 ml of water and 3.4 ml of ethanol were added in the fashion presented in the preparation procedure followed by drying and perforating the resultant film. The formed films were uniform, clear and transparent in appearance and able to dissolve rapidly with higher drug loading.

The films from Example 9 were also cast to result into various dry film thicknesses as following:

### Trial 1

10×12 cm film was cast to get a film thickness of about 150 μm.

### Trial 2

10×12 cm film was cast to get a film thickness of about 225 μm.

### Trial 3

10×12 cm film was cast to get a film thickness of about 300 μm.

### In Vivo Dissolution Trials

Six 2-cm² films were cut from each film; these were divided into two groups. In one group of three films, 9 evenly spaced 2 mm diameter holes were punctured by means of a hollow tube. These films were placed on the center of tongue and gentle motion of the tongue was provided by slightly moving the film against the upper palate. The time of complete dissolution was noted for the films with and without perforations. The dissolution data is shown below in tables 2 to 4. The film with and without perforations is shown in FIG. 4.

TABLE 2
Dissolution times for 150 μm film

| | |
|---|---|
| Trial 1 (150 μm) | Time for dissolution (seconds) |

| Sr No | Perforations | Control (no perforations) |
|---|---|---|
| 1 | 46 | 64 |
| 2 | 30 | 60 |
| 3 | 42 | 61 |
| Avergae | 39.33 | 61.67 |
| Std Dev | 8.33 | 2.08 |

TABLE 3
Dissolution times for 225 μm film

| | |
|---|---|
| Trial 3 (225 μm) | Time for dissolution (seconds) |

| Sr No | Perforations | Control (no perforations) |
|---|---|---|
| 1 | 62 | 120 |
| 2 | 60 | 90 |
| 3 | 50 | 94 |
| Avergae | 57.33 | 101.33 |
| Std Dev | 6.43 | 16.29 |

TABLE 4
Dissolution times for 300 μm film

| | |
|---|---|
| Trial 2 (300 μm) | Time for dissolution (secconds) |

| Sr No | Perforations | Control (no perforations) |
|---|---|---|
| 1 | 92 | 114 |
| 2 | 85 | 101 |
| 3 | 93 | 135 |
| Avergae | 90 | 116.67 |
| Std Dev | 4.36 | 17.16 |

1. A oral fast dispersing or dissolving system for administration of drugs comprising a polymer based matrix system, suitable plasticizer, buffer agents, sweetners, emulsifiers and drug) formed into film with physically made perforations into the body of the strip.

2. The system in claim 1 wherein the perforations are made into the film by means of mechanical drilling with pins, needles or laser drilling or burning or any other method of creating perforations.

3. The size, shape, number and volume of the perforations in the film in claim 1 are variable.

4. The perforated film in claim 1 can accommodate drugs from various pharmacological categories but not restricted to one class of drugs either from conventional small molecules or proteins or peptides.

5. The drug concentrations in claim 1 range from few micro grams to few hundred milligrams, more specifically from 10 micrograms to 500 milligrams. For some applications such as wound healing/dressing or personal hygiene or transdermal in situ applications the drug concentration can be from few milligrams to few grams, specifically 1 milligram to 10 grams.

6. The perforated film dosage form in claim 1 comprises water soluble polymer/s wherein the water-soluble hydrocolloid is a polymer selected from the group consisting of a natural, semi-natural and synthetic biopolymer.

7. The perforated film dosage form in claim 1, wherein the water-soluble hydrocolloid is selected from the group consisting of a polysaccharide and a polypeptide.

8. The perforated film dosage form in claim 6, wherein the water-soluble hydrocolloid comprises a hydroxypropylmethylcellulose polymer.

9. The perforated film dosage form in claim 8, wherein the hydroxypropylmethylcellulose polymer has a molecular weight of less than 250,000 Daltons, preferably having methoxy content of about 19-30% and hydroxypropyl content of 7-12%.

10. The perforated film dosage form of claim 1, wherein the film further comprises at least one of an emulsifier, a plasticizer, a taste modifying agent, a water soluble inert filler, a preservative, a coloring agent, a stabilizer, a disintegrant, effervescence agent and a buffering agent.

11. The perforated film dosage form of claim 1, wherein the film further comprises an emulsifier at a concentration in the range of 0 to 10% w/w of the unit dosage form.

12. The perforated dosage form in claim 1 also further comprises at least one of a sweetening agent in the concentration range of 0.1 to 10% w/w, a flavoring agent in the concentration range of 0.1 to 5% w/w, a taste masking agent in the concentration range of 0.01 to 2% w/w and a coloring agent of 0.01 to 4% w/w of the unit dosage form.

13. The perforated film dosage form in claim 1, wherein the film further comprises a water soluble inert filler present at a concentration in the range of 0 to 50% w/w of the unit dosage form.

14. The perforated film dosage form in claim 1, wherein the film further comprises a preservative present at a concentration in the range of 0.01 to 10% w/w of the unit dosage form.

15. The perforated film dosage form in claim 1, wherein the active agent is present at a concentration in the range of 0.01 to 80% w/w of the unit dosage form.

16. The perforated film dosage form in claim 1, in addition to hydrocolloids and the active agents, may contain any or all of the following ingredients: emulsifying agents, solubilizing agents, wetting agents, suspending agents, taste modifying agents, plasticizers, active agents, water soluble inert fillers, preservatives, buffering agents, effervescence agents, coloring agents, and stabilizers. In a preferred unit dosage form, the percentage dry weight concentration of at least single ingredients incorporated in a film in each of the following categories is as follows: emulsifying agent (0.1%-10%), plasticizer (0.5-20%), active agents (0.01-80%), taste modifying agents (0.1-10%), coloring agents (0.01-5%), water soluble inert fillers (0.5-50%), preservatives (0.01-10%), buffering agents (0.1-10%) and stabilizers (0.01-5%).

17. The perforated film dosage form in claim 1, wherein the sexual dysfunction active agent is Sildenafil Citrate.

18. The perforated film dosage form in claim 1, wherein the film has a dry film thickness in the range of 0.050 to 20 mil.

19. The perforated film dosage form in claim 18, wherein the film has a dry film thickness of less than 10 mils.

20. The perforated film dosage form of claim 1, wherein the film exhibits a dissolution time in the range of 1 to 600 seconds upon application.

21. The perforated film dosage form of claim 1, wherein the film exhibits a dispersion/dissolution time in the range of 1 to 300 seconds upon application to mucosal surface or skin.

22. The perforated film dosage form of claim 1, wherein the bioactive agent is encapsulated within a polymer, wherein the polymer is chemically or physically similar or distinct from the hydrocolloid to result in controlled dissolution of bioactive agent in relation to the film.

23. The perforated film dosage form of claim 1, wherein the dosage unit comprises at least two active agents.

24. The perforated film dosage form in claim 1 are used in various drug delivery applications including oral delivery followed by GI absorption, oral cavity application for buccal absorption, oral cavity local treatment purposes, periodontal applications, sublingual applications, skin care and wound treatment application.

25. The perforated film dosage form in claim 1 are also used as wound dressing/healing preparations, personal hygiene and intra vaginal or urethral delivery of bioactive agents.

26. The perforated film dosage form in claim 1 can be in any shape including square, rectangle, round, oval, zig zag, oblong, elongated, twisted, ribbon shaped, triangle shaped, and other imaginable shapes and sizes depending on the need and type of drug delivery but not restricted to one size or shape or type.

27. The perforated film dosage form in claim 1 even at low drug loads disperse or dissolve faster than their counterparts without perforations.

28. The perforated film dosage form in claim 1 wherein the films are made with water insoluble polymers resulting in faster disintegration of the films.

29. The perforated film dosage form in claim 1 wherein the films are made with pH dependent polymers resulting in faster disintegration of the films.

30. The perforated film dosage form in claim 1 wherein the films are made using polymers normally not employed in such fast disintegrating films.
